(19) United States
(12) Patent Application Publication
(10) Pub. No.: US 2004/0053401 A1
(43) Pub. Date: Mar. 18, 2004

(54) Preparation of enantiomerically pure hydroxy esters and acids

(76) Inventors: Reinhold Ohrlein, Rheinfelden-Herten (DE); Gabriele Baisch, Binzen (DE)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 10/416,383

(22) PCT Filed: Nov. 6, 2001
(86) PCT No.: PCT/EP01/12821

(30) Foreign Application Priority Data
Nov. 14, 2000 (EP) 008110744

(51) Int. Cl. C07C 67/31; C07C 69/732; C07C 69/738; C12P 41/00

(52) U.S. Cl. 435/280

(57) ABSTRACT

The present invention relates to a process for the preparation of compounds of formula (1), wherein R₁ is unsubstituted or substituted C₁-C₈alkyl or a radical of formula —COOR₃, wherein R₃ is hydrogen or unsubstituted or substituted C₁-C₈alkyl, R₂ is hydrogen or unsubstituted or substituted C₁-C₈alkyl, X is the radical —O— or —NH—, Y is hydrogen or an acyl or silyl radical, n is the number 0, 1 or 2, and the chiral carbon atom denoted by the symbol* in the compound of formula (1) is predominantly in pure form in either the R or S configuration, in which process a compound of formula (2) is converted by enantioselective hydrogenation and, where approriate, introduction of the radical Y to form an enantiomeric mixture, enriched with one of the enantiomers (R or S configuration), of the compound of formula (3), and the enantiomeric mixture is separated by enzymatic stereoselective hydrolysis, alcoholysis, aminolysis or ammonolysis, and in the case of the preparation of compounds of formula (1) wherein X is the radical —NH—, the resolution is effected by enzymatic stereoselective aminolysis or ammonolysis in the presence of a compound of formula NH₂—R₂′, wherein R₂′ is as defined above for R₂.

The present invention relates to a process for the preparation of enantiomerically pure hydroxy esters and of the corresponding acids by combined hydrogenolytic and enzymatic synthesis.

The following compounds of formula (1) are inter alia important intermediates in the preparation of pharmaceuticals or insecticides.

For example, 2-hydroxybutyric acid esters are inter alia important intermediates in the preparation of pharmacologically active ACE inhibitors (ACE: Angiotensin Converting Enzyme).

ACE inhibitors belong to the active ingredient group of antihypertonics and, following oral administration, bring about competitive inhibition of the so-called angiotensin converting enzyme and thus a lowering of blood pressure. Especially preferred 2-hydroxybutyric acid esters have the R configuration.

An important active ingredient is 3-[(1-(ethoxycarbonyl)-3-phenyl-(1S)-propyl)amino]-2,3,4,5-tetrahydro-2-oxo-1H-1-(3S)-benzazepine-1-acetic acid hydrochloride, which is known by the INN benazepril hydrochloride and is commercially available in various forms for oral administration, e.g. tablets, under the name Cibacen® (trademark of Novartis AG, Basle, Switzerland).

2-Hydroxybutyric acid esters can also be used as intermediates in the preparation of other known ACE inhibitors, e.g. enalapril, cilazapril, spirapril, quinapril, ramipril and lisinopril (INNs). 2-Hydroxybutyric acid esters can also be used in the synthesis of various types of insecticide.

Furthermore, 4-chloro-3-hydroxybutanoates, for example, are used as pharmaceutical intermediates in the synthesis of L-carnitine (vitamin B_{T}), antieleptics or cholesterol biosynthesis inhibitors (HMG-CoA reductase inhibitors).

In the case of malic acids, for example, both enantiomers are used in synthesis. They are used inter alia as auxiliary reagents in the separation of racemates, for which reason they need to be available inexpensively in large amounts. Malic acids are also versatile constituents of various pyrones, coumaric acids, paclitaxel side chains and insecticides.

WO-A-99/50223 discloses a process for the preparation of 2-hydroxybutyric acid esters by stereoselective hydrogenation of the corresponding diketo compounds. The enantiomers are separated in that case by customary processes, for example by crystallisation from a suitable solvent. Such a procedure does not, however, satisfy the demands made in terms of both the yields and the purity of the desired enantiomers. By combining stereoselective hydrogenation with enzymatic separation of the enantiomers, a process has now been found by means of which surprisingly the desired enantiomers can be obtained in high yields with high optical purity.

The present invention accordingly relates to a process for the preparation of compounds of formula

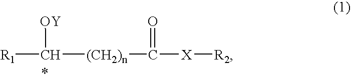

wherein

R₁ is unsubstituted or substituted C₁-C₈alkyl or a radical of formula —COOR₃, wherein R₃ is hydrogen or unsubstituted or substituted C₁-C₈alkyl,

R₂ is hydrogen or unsubstituted or substituted C₁-C₈alkyl,

X is the radical —O— or —NH—,

Y is hydrogen or an acyl or silyl radical,

n is the number 0, 1 or 2, and

the chiral carbon atom denoted by the symbol * in the compound of formula (1) is present predominantly In pure form in either the R or S configuration,

in which process a compound of formula

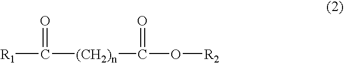

is converted by enantioselective hydrogenation and, where appropriate, introduction of the radical Y to form an enantiomeric mixture, enriched with one of the enantiomers (R or S configuration), of the compound of formula

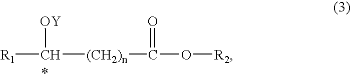

and the enantiomeric mixture is separated by enzymatic stereoselective hydrolysis, alcoholysis, aminolysis or ammonolysis,

and in the case of the preparation of compounds of formula (1) wherein X is the radical —NH—, the resolution is effected by enzymatic stereoselective aminolysis or ammonolysis in the presence of a compound of formula NH₂—R₂′, wherein R₂′ is as defined above for R₂.

As unsubstituted or substituted C₁-C₈alkyl for R₁ there come into consideration especially corresponding C₁-C₄alkyl radicals, preferably corresponding methyl or ethyl radicals. Examples of substituents of the alkyl radicals that may be mentioned are halogen or unsubstituted or further-substituted phenyl or benzoyl. When halogen substituents are present, they are, in this case and hereinafter, especially chlorine or bromine, preferably chlorine. The phenyl and benzoyl radicals mentioned as substituents may be unsubstituted or substituted, for example by C₁-C₄alkyl, C₁-C₄alkoxy, C₁-C₄alkylamino, C₁-C₄alkanoyl, amino, nitro or by halogen, especially by C₁-C₄alkyl, C₁-C₄alkoxy or by halogen. The phenyl radical is preferably unsubstituted. The benzoyl radical Is preferably unsubstituted or substituted by chlorine.

As unsubstituted or substituted C₁-C₈alkyl for R₃ there come into consideration, for example, the alkyl radicals mentioned above for R₁. As substituents of the alkyl radicals, special mention may be made of unsubstituted or further-substituted phenyl radicals. The phenyl radical can be substituted as indicated above in the case of R₁. The phenyl radical is preferably unsubstituted.

R₁ is preferably C₁-C₈alkyl that is unsubstituted or substituted by halogen or by phenyl or benzoyl that are unsubstituted or further substituted by C₁-C₄alkyl, C₁-C₄alkoxy, C₁-C₄-alkylamino, C₁-C₄alkanoyl, amino, nitro or by halogen; or

a radical of formula —COOR₃, wherein R₃ is hydrogen or unsubstituted or phenyl-substituted C₁-C₈alkyl and the phenyl radical is unsubstituted or further substituted by C₁-C₄alkyl, C₁-C₄-alkoxy, C₁-C₄alkylamino, C₁-C₄alkanoyl, amino, nitro or by halogen.

R₁ is especially C₁-C₈alkyl that is unsubstituted or substituted by halogen or by phenyl or benzoyl that are unsubstituted or further substituted by C₁-C₄alkyl, C₁-C₄alkoxy, C₁-C₄-alkylamino, C₁-C₄alkanoyl, amino, nitro or by halogen. Preferred substituents of the phenyl and benzoyl radicals are C₁-C₄alkyl, C₁-C₄alkoxy or halogen (e.g. chlorine).

R₁ is especially preferably methyl or ethyl, each of which is unsubstituted or substituted by halogen or by phenyl or benzoyl that are unsubstituted or further substituted by C₁-C₄alkyl, C₁-C₄alkoxy or by halogen. Of particular interest are radicals R₁ that are methyl or ethyl unsubstituted or substituted by chlorine, phenyl or by benzoyl that is unsubstituted or further substituted by chlorine.

As unsubstituted or substituted C₁-C₈alkyl for R₂ there come into consideration, for example, the alkyl radicals mentioned above for R₁. As substituents of the alkyl radicals, special mention may be made of unsubstituted or further-substituted phenyl radicals. The phenyl radical can be substituted as indicated above in the case of R₁. The phenyl radical is preferably unsubstituted. R₂ is preferably hydrogen, C₁-C₄alkyl or benzyl, especially C₁-C₄-alkyl or benzyl. Examples of radicals R₂ that may be mentioned are methyl, ethyl and benzyl. Special preference is given to methyl and especially ethyl.

X is preferably the radical —O—.

Y as an acyl radical is, for example, a radical of formula —C(O)—R₄ wherein R₄ is unsubstituted or phenyl-substituted C₁-C₈alkyl. R₄ is preferably unsubstituted or phenyl-substituted C₁-C₄-alkyl, especially unsubstituted or phenyl-substituted methyl or ethyl. Acetyl is especially preferred.

Y as a silyl radical is, for example, a radical of formula —Si(R₅)₃, wherein the substituents R₅ can have identical or different meanings and are unsubstituted or phenyl-substituted C₁-C₅-alkyl. R₅ is preferably unsubstituted C₁-C₈alkyl, especially C₁-C₄alkyl and preferably methyl or tert-butyl.

Y is preferably an acyl radical.

n is preferably the number 0 or 1, especially the number 1.

The expression “predominantly in pure form”, in the context of formula (1), means an enantiomeric distribution that departs from the 50/50 distribution of a racemate in that it is from 95/5 to 100/0, especially from 97.5/2.5 to 100/0 and preferably from 99/1 to 100/0 in favour of the R or S configuration. The enantiomeric distribution is especially preferably from 99.5/0.5 to 100/0.

For compounds of formula (1) wherein R₁ is alkyl substituted by unsubstituted or further-substituted benzoyl, the S configuration is preferred. In the other cases, the R configuration is preferred.

The symbols and

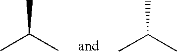

in the structural formulae indicate that a predominant number of the molecules have the indicated stereochemical configuration at the centre of chirality, the configuration being denoted by either R or S in accordance with the rules of nomenclature (R,S nomenclature) of Kahn, Ingold and Prelog.

The compounds of formula (2) are to be understood as including also the corresponding tautomeric forms thereof.

The compounds of formula (2) are known or can be obtained analogously to known processes.

The enantioselective hydrogenation is carried out preferably using platinum as catalyst in the presence of a chiral modifier, especially in the presence of a cinchona alkaloid as chiral modifier (see e.g. WO-A-99/50223).

A chiral modifier contains a basic nitrogen atom located near one or more centres of chirality, which are in turn bonded to a bicyclic aromatic group. Suitable chiral modifiers are described by A. Pfaltz and T. Heinz in Topics in Catalysis 4(1997) 229-239. Preference is given to cinchona alkaloids, which are known by that name and belong to the group of quinoline plant bases that can be isolated chiefly from the bark of trees of the Cinchona and Remijia families. That definition includes in particular the alkaloids (−)-quinine, (+)-quinidine, (+)-cinchonine and (−)-cinchonidine. The use of (−)-quinine and (−)-cinchonidine results in compounds (3) in the R form whereas, when (+)-quinidine and (+)-cinchonine are used, compounds (3) in the S form are obtained. The use of (−)-cinchonidine and derivatives thereof is preferred.

In an especially preferred embodiment, the chiral modifiers used are derivatives of the cinchonidine of formula

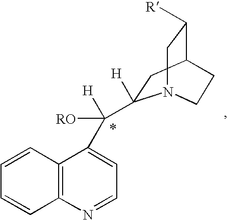

wherein R is hydrogen, methyl, acetyl, lactoyl or benzyl-etherified lactoyl and R′ is ethyl or hydroxymethyl, and the chiral centre is indicated by the symbol *.

The above-mentioned compound in which R is hydrogen and R′ is ethyl is known as 10,11-dihydrocinchonidine (HCd), the compound in which R is methyl and R′ is ethyl is known as O-methoxy-10,11-dihydrocinchonidine (MeOHCd) and the compound in which R is hydrogen and R′ is hydroxymethyl is known as norcinchol.

In a preferred embodiment of the process, 10,11-dihydrocinchonidine (HCd) is used as chiral modifier.

The enantioselective reduction is carried out in a manner known per se. The platinum catalysts used may be present in the form of so-called polymer-stabilised colloidal metal clusters, e.g. as described by X. Zuo et al. in Tetrahedron Letter 39(1998) 1941-1944, or are preferably applied to suitable carriers. Examples of suitable carriers are carbon, aluminium oxide, silicon dioxide, Cr₂O₃, zirconium dioxide, zinc oxide, calcium oxide, magnesium oxide, barium sulfate, calcium carbonate and aluminium phosphate. Preference is given to aluminium oxide. The catalysts are activated in a manner known per se with hydrogen at about from 200 to 400° C. and then modified by impregnation with the solution of the cinchona alkaloid, and/or the cinchona alkaloid is added directly during the reduction reaction.

Hydrogenation is carried out in the presence of water or an organic solvent. Preference is given to the use of polar and non-polar aprotic solvents or polar protic solvents or mixtures thereof.

Examples of suitable non-polar aprotic solvents are hydrocarbons, for example aliphatic hydrocarbons, e.g. hexane, heptane or petroleum ether, cycloaliphatic hydrocarbons, e.g.

cyclohexane or methylcyclohexane, aromatic hydrocarbons, e.g. benzene, toluene or xylene.

Examples of suitable polar aprotic solvents are ethers, for example aliphatic ethers e.g. diisopropyl ether, 1,2-diethoxyethane or tert-butyl methyl ether, cyclic ethers, e.g. tetrahydrofuran or dioxane, amides, e.g. dimethylformamide or N-methylpyrrolidone. Particularly suitable are ethers, especially tetrahydrofuran.

Examples of suitable polar protic solvents are, for example, alcohols, e.g. ethanol or n-butanol.

The process can be carried out preferably in the liquid phase discontinuously or continuously, especially using a catalyst suspension as liquid phase hydrogenation or in a bubble column or using a shaped catalyst in a trickle bed. The reaction can also be carried out in the gaseous phase using a pulverulent catalyst in a fluidized bed or using a shaped catalyst in a solid bed.

The hydrogenation can be carried out within wide temperature ranges. Temperatures of from room temperature to about 100° C., especially from 20° to about 50° C., have proved advantageous.

The hydrogen pressure in the hydrogenation can vary within wide ranges, for example from 1 to 200 bar, preferably from 5 to 100 bar, especially from 10 to 60 bar. The hydrogen pressure used depends substantially on the hydrogenating equipment available.

The reaction time can vary within wide limits. It is dependent upon the catalyst used, the hydrogen pressure, the reaction temperature and the equipment used. It can be, for example, from half an hour to 24 hours. Reaction times of about from half an hour to two hours are advantageous.

The reaction products are isolated according to known methods, for example by filtration and removal of the solvent by evaporation.

The enantiomeric mixture of the compound of formula (3), enriched with one of the enantiomers by hydrogenation, preferably has an enantiomeric distribution of from 65/35 to 95/5, especially from 70/30 to 95/5 and preferably from 75/25 to 95/5 in favour of the R or S configuration. Special preference is given to an enantiomeric distribution of from 80/20 to 95/5.

The enzymatic separation can be carried out, for example, according to the following schemes:

a) an enantiomeric mixture, enriched with one of the enantiomers, of a compound of formula (3) wherein R₂ is unsubstituted or substituted C₁-C₈alkyl is converted by enzymatic hydrolysis to form a mixture of the compounds of formulae

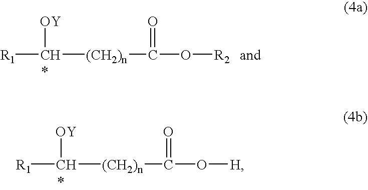

wherein

R₂ is as defined above,

R₁, Y and n are as defined for formula (1), and one of the compounds of formulae (4a) and (4b) is in the R configuration and the other of the compounds of formulae (4a) and (4b) is in the S configuration;

b) an enantiomeric mixture, enriched with one of the enantiomers, of a compound of formula (3) wherein Y is an acyl radical is converted by enzymatic hydrolysis to form a mixture of the compounds of formulae

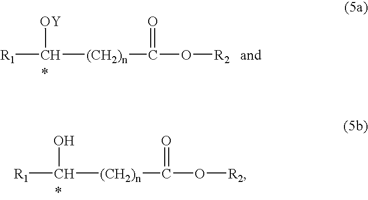

wherein

Y is as defined above,

R₁, R₂ and n are as defined for formula (1), and

one of the compounds of formulae (5a) and (5b) is in the R configuration and the other of the compounds of formulae (5a) and (5b) is in the S configuration;

c) an enantiomeric mixture, enriched with one of the enantiomers, of a compound of formula (3) wherein R₂ is unsubstituted or substituted C₁-C₈alkyl and Y is an acyl radical is converted by enzymatic hydrolysis to form a mixture of the compounds of formulae

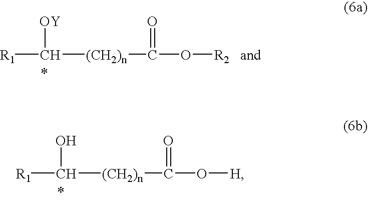

wherein

R₂ and Y are as defined above,

R₁ and n are as defined for formula (1), and

one of the compounds of formulae (6a) and (6b) is in the R configuration and the other of the compounds of formulae (6a) and (6b) is in the S configuration;

d) an enantomeric mixture, enriched with one of the enantiomers, of a compound of formula (3) is converted by enzymatic aminolysis or ammonolysis in the presence of a compound of formula NH₂—R₂′ to form a mixture of the compounds of formulae

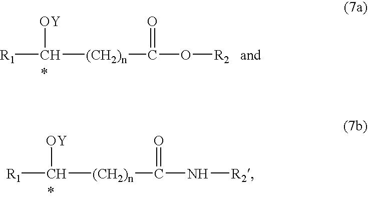

wherein

R₁, R₂, R₂′ and n are as defined for formula (1), and

one of the compounds of formulae (7a) and (7b) is in the R configuration and the other of the compounds of formulae (7a) and (7b) is in the S configuration.

Preference is given to process variants a), b) and c), especially a) and c), preferably c).

In process variants a), b) and c) it is also possible additionally to add a compound of formula HO—R₂′, there being obtained, as a result of alcoholysis, compounds which correspond to the compounds of formulae (4b), (5b) and (6b), respectively, but which contain the radical —COOR₂′ instead of the radical —COOR₂ or —COOH. The definitions and preferred meanings given above for R₂ apply also to the radical R₂′.

If desired, the products can be purified again by recrystallisation in order to increase the enantiomeric purity further.

The introduction of a radical Y can be carried out according to known processes, for example by acylation.

Suitable as enzymes, especially as hydrolytic enzymes, are especially esterases, lipases and proteases (amidases) (in this connection see also U. T. Bornscheuer, R. T. Kazlauskas in: Hydrolases in Organic Synthesis; Wiley-VCH, 1999, pages 65-195, ISBN 3-527-30104-6).

As esterases there may be mentioned, for example, esterases from animals (e.g. PLE), from microorganisms or from fungi (e.g. B. subtilis esterase, Pichia esterases, yeast esterases, Rhizopus sp. esterases, Penicillium sp. esterases).

As lipases there may be mentioned, for example, lipases from animals (e.g. PPL), fungi and microorganisms (G. candidum (GCL), H. lanuginosa (HLL), Rhizopus sp. (RML, ROL), Candida sp. (CAL-A, CAL-B, CCL), Aspergillus sp.(ANL), Pseudomonas sp. (PCL, PFL).

As proteases there may be mentioned, for example, subtilisin, thermitase, chymotrypsin, thermolysin, papain, aminoacylases, penicillin amidases and trypsin.

The use according to the invention of biocatalysts is not, of course, limited to the enzymes listed. The enzymes can be used according to the invention for stereoselective hydrolysis, alcoholysis, aminolysis or also ammonolysis.

The enzymes can be obtained as crude isolates and/or in purified form from natural sources and/or from microorganisms by modern cloning processes, such as over-expression and amplification. The enzymes are also commercially available. Suitable enzymes are obtainable, for example, from the companies Fluka, Sigma, Novo, Amano, Roche. There may also be mentioned the enzymes listed in current literature (in this connection see, for example, H.-J. Rehm, G. Reed in Biotechnology, VCH 1998, 2^{nd} Ed., pages 40-42).

The enzymes can be used as such or immobilised or adsorbed on various carriers, such as silica gel, Celite, Eupergit, etc. or as so-called CLEC's (crosslinked enzymes), as supplied by the company ALTUS BIOLOGICS, use not being limited, of course, to the list given (in this connection see also: U. T. Bornscheuer, R. T. Kazlauskas in Hydrolases in Organic Synthesis, Wiley-VCH, 1999, pages 61-64, ISBN 3-527-30104-6; K. Faber in Biotrans-formation in Organic Chemistry, Springer 1997, 3^{rd} Ed., 345-357, ISBN 3-540-61688-8; H.-J. Rehm, G. Reed in Biotechnology, VCH 1998, 2^{nd} Ed., 407-411).

The enzymes can be used in purely organic solvents, such as hexane, toluene, benzene, tetrahydrofuran, diethyl ether, methyl tert-butyl ether, methylene chloride, etc., or in mixtures of such solvents with water or with aqueous buffer solutions. It is customary for the aqueous phase to be buffered (e.g. pH=5-9), it being possible to use customary buffers (in this connection see also K. Faber in Biotransformation in Organic Chemistry, Springer 1997, 3^{rd} Ed., 305; U. T. Bornscheuer, R. T. Kazlauskas in: Hydrolases in Organic Synthesis, Wiley-VCH, 1999, pages 61-65). The pH value is kept constant during the reaction; most suitable for the purpose is an automatic titrator having a set base or acid solution. The reaction temperature is, for example, in the range of from 10 to 50° C., preferably from 25 to 40° C. The amount of biocatalyst used and the concentrations of the reagents can vary within wide limits and may be selected according to the substrate and the reaction conditions chosen in each case.

If a high enantiomeric purity is obtained as a result of the stereoselective hydrogenation, with the result that large amounts of the desired main enantiomer can be separated off just by crystallisation, the above procedure is also excellently suitable for obtaining the remainder of the desired enantiomer from the mother liquors. In that manner it is possible to obtain very high yields of the desired enantiomer.

The following Examples illustrate the invention:

### EXAMPLE 1

a) Hydrogenation

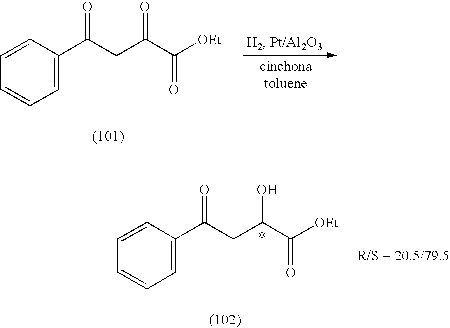

2.0 g of the compound of formula (101) (MW 220.24) are dissolved with 30 ml of toluene in a 50 ml stainless steel autoclave having a double-wall jacket, magnetic stirrer and current-breaker. 50 mg of 5% Pt/Al₂O₃ (Engelhard 4759, pretreated for 2 h in H₂ at 400° C.) and 5 mg of (+)-10,11-dihydrocinchonidine are added thereto. The autoclave is closed and flushed twice with argon and twice with hydrogen. A pressure of 60 bar H₂ is then applied and the reaction is started by stirring with the magnetic stirrer (1200 rpm). During the reaction a temperature of 25° C. (thermostat) and a pressure of 60 bar H₂ are maintained. After a reaction time of 160 min., the take-up of hydrogen has ceased. Once the pressure has been released, the autoclave is flushed again with argon. The reaction mixture is filtered and concentrated by evaporation to yield the compound of formula (102) [MW 222.24; ratio of configurations R/S=20.5/79.5].

b) Enzymatic Separation

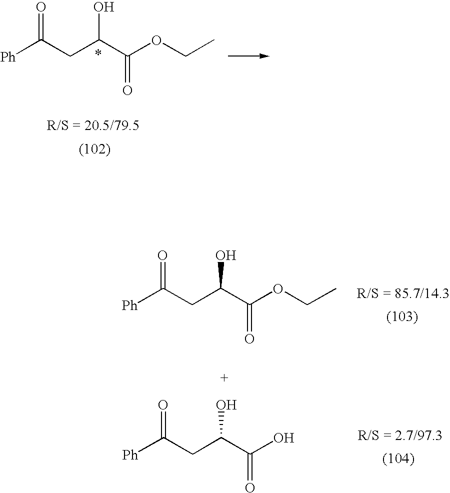

5.0 g of the compound of formula (102) obtained by hydrogenation are suspended in 43 ml of water together with 4.8 ml of 0.1 M phosphate buffer (pH=7). 75 mg of lipase PS (AMANO) are added thereto and the mixture is stirred vigorously at room temperature. The pH is kept at from 6.9 to 7.2 by means of 0.5N sodium hydroxide solution (theoretical consumption 39.14 ml). The protein is then filtered off over Celite. The aqueous solution is subsequently extracted with ethyl acetate, and the organic phase is separated off and back-extracted with saturated sodium chloride solution. 1.09 g of the compound of formula (103) are obtained from the organic phase after removal of the solvent by evaporation. The aqueous phase is adjusted to pH=1-2 with 2N hydrochloric acid and is also extracted with ethyl acetate. After removal of the solvent, 3.06 g (88%) of the desired compound of formula (104) are obtained in the form of a white solid.

¹H-NMR(CDCl₃) of the compound of formula (104): 7.87 (m, 2H), 7.53 (m, 1H), 7.40 (m, 2H), 4.61 (dd, J=3.81 and 5.86 Hz; 1H), 3.47 (dd, J=3.81 and 17.59 Hz; 1H), 3.37 (dd, J=5.86 and 17.59 Hz; 1H).

In order to determine the enantiomeric ratio of the compound of formula (104), the acid is esterified by means of (trimethylsilyl)diazomethane according to customary methods without racemisation.

By means of HPLC (Chiracel AD), a ratio of R/S=2.7/97.3 is determined.

Retention time R isomer: 48.15 min;

Retention time S isomer: 42.88 min.

### EXAMPLE 2

a) Hydrogenation

The hydrogenation is carried out as indicated in Example 1a).

b) Acetylation

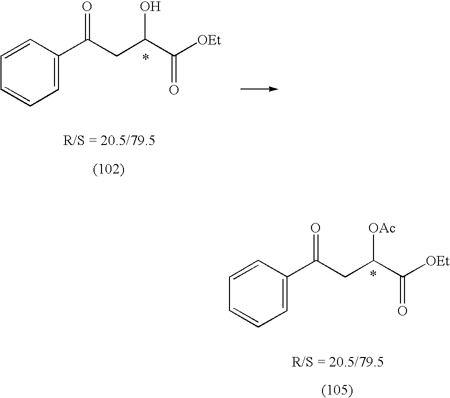

10.0 g of the compound of formula (102) obtainable according to Example 2a) are dissolved at 0° C. in methylene chloride; 3.7 g of acetyl chloride and 3.8 ml of pyridine are added thereto and the mixture is stirred until the conversion is complete. The mixture is diluted with ethyl acetate and washed, in succession, with 1 N hydrochloric acid, saturated sodium hydrogen carbonate solution and saturated sodium chloride solution, and the organic phase is dried over sodium sulfate. Removal of the solvent yields 11.5 g (97%) of the compound of formula (105).

¹H-NMR(CDCl₃) of the compound of formula (105): 7.93 (m, 2H), 7.59 (m, 1H), 7.48 (m, 2H), 5.68 (dd, J=4.10 and 5.86 Hz; 1H), 4.22 (q, J=7.03 Hz; 2H, 3.57 (dd, J=7.91 and 17.29 Hz; 1H), 3.47 (dd, J=3.82 and 17.30 Hz; 1H), 2.08 (s, 3H), 1.27 (t, J=7.03 Hz, 3H).

c) Enzymatic Separation

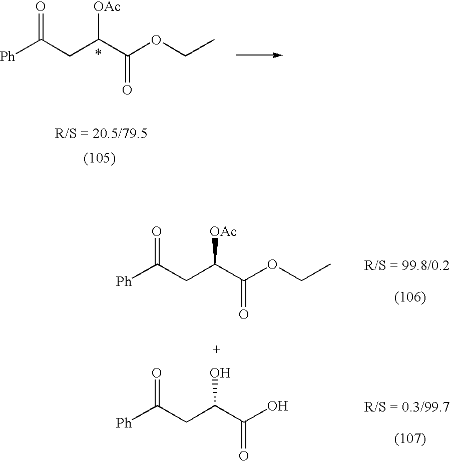

1.0 g of the compound of formula (105) obtainable according to Example 2b) are suspended in 50 ml of a mixture of hexane/toluene (ratio by volume 4/1) together with 25 ml of 1 M phosphate buffer (pH=7). 250 mg of lipase PS (AMANO) are added thereto and the mixture is stirred vigorously at room temperature. After about 28 hours, the protein is filtered off over Celite. The aqueous phase is then separated off from the organic phase, adjusted to pH=1-2 with 2N hydrochloric acid and extracted with ethyl acetate. After removal of the solvent, 0.54 g (93%) of the desired compound of formula (107) is obtained in the form of a white solid. In addition, 0.2 g (100%) of the compound of formula (106) is recovered from the toluene phase.

### EXAMPLE 3

a) Hydrogenation

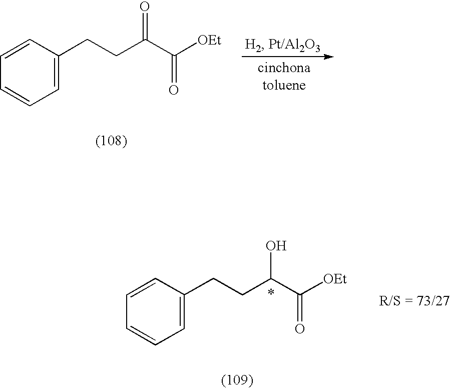

The hydrogenation is carried out analogously to the instructions in Example 1a).

b) Enzymatic Separation

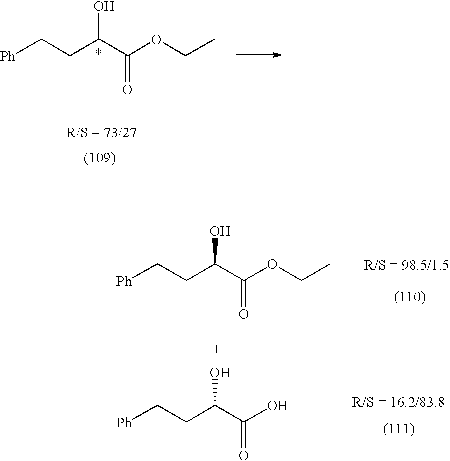

5.0 g of the compound of formula (109) obtainable according to Example 3a) are suspended in 43 ml of water together with 4.8 ml of 0.1 M phosphate buffer (pH=7). 100 mg of lipase PS (AMANO) are added thereto and the mixture is stirred vigorously at room temperature. The pH is maintained at from 6.9 to 7.2 by means of 0.5N sodium hydroxide solution (theoretical consumption 39.14 ml). The protein is then filtered off over Celite. The aqueous solution is subsequently extracted with ethyl acetate, and the organic phase is separated off and back-extracted with saturated sodium chloride solution. 3.46 g (95%) of the desired compound of formula (110) are obtained from the organic phase after removal of the solvent by evaporation.

¹H-NMR(CDCl₃) of the compound of formula (110): 7.29 (m, 2H), 7.21 (m, 2H), 4.23 (q, J=7.33 Hz, 2H), 4.17 (dd, J=4.10 and 7.62 Hz; 1H), 2.77 (m, 1H), 2.10 (m, 2H), 1.96 (m, 2H), 1.29 (t, J=7.33 Hz, 3H).

By means of HPLC (Chiracel OD), a ratio of R/S=98.5/1.5 is determined for the compound of formula (110).

Retention time R isom r: 7.38 min;

Retention time S isomer: 6.26 min.

### EXAMPLE 4

a) Hydrogenation

The hydrogenation is carried out as indicated in Example 3a).

b) Acetylation

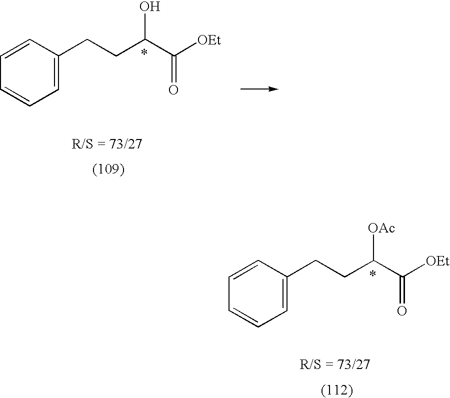

The acetylation is carried out analogously to the instructions in Example 2b). The acetylated enantiomers are analysed on Chiracel AD.

Retention time R isomer: 4.61 min;

Retention time S isomer: 5.55 min.

¹H-NMR(CDCl₃) of the compound of formula (112): 7.29 (m, 2H), 7.21 (m, 3H), 5.99 (d, J=6.65, 1H), 4.18 (q, J=7.03 Hz; 2H), 2.74 (dd, J=7.62 and 9.96 Hz; 2H), 2.15 (m, 1H), 2.10 (s, 3H), 1.27 (t, J=7.03 Hz, 3H).

c) Enzymatic Separation

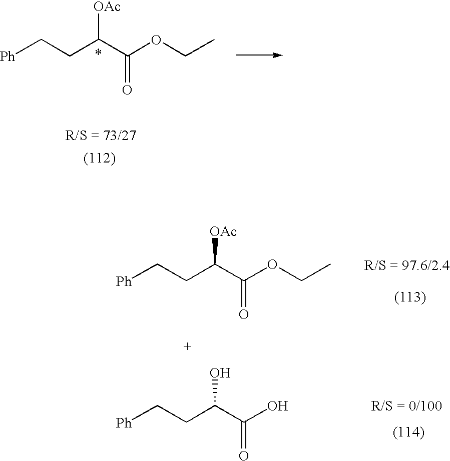

1.0 g of the compound of formula (112) obtainable according to Example 4b) are suspended in 50 ml of hexane together with 25 ml of 1 M phosphate buffer (pH=7). 400 mg of lipase PS (AMANO) are added thereto and the mixture is shaken at room temperature. After about 32 hours, the protein is filtered off over Celite. The aqueous phase is then separated off from the organic phase and washed with saturated sodium chloride solution. Concentration by evaporation of the solvent yields 0.659 (89%) of the desired compound of formula (113).

The aqueous phase is adjusted to pH=1-2 with 2N hydrochloric acid and extracted with ethyl acetate. After removal of the solvent, 0.15 g (79%) of the compound of formula (114) is obtained in the form of a white solid.

### EXAMPLE 5

a) Hydrogenation

The hydrogenation is carried out analogously to the instructions in Example 1a).

b) Acetylation

The acetylation is carried out analogously to the instructions in Example 2b).

c) Enzymatic Separation

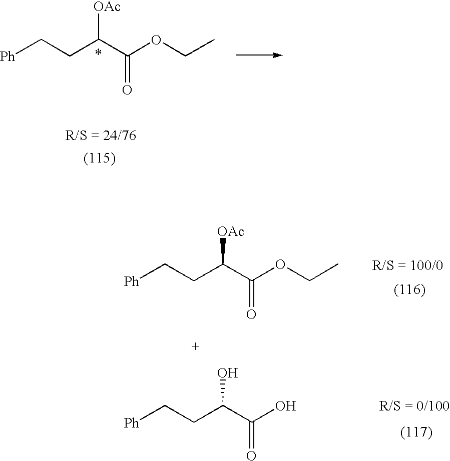

1.0 g of the compound of formula (115) obtainable according to Steps a) and b) is suspended in 50 ml of hexane together with 25 ml of 1 M phosphate buffer (pH=7). 400 mg of lipase PS (AMANO) are added thereto and the mixture is shaken at room temperature. After about 32 hours, the protein is filtered off over Celite. The aqueous phase is then separated off from the organic phase and washed with saturated sodium chloride solution. Concentration by evaporation of the solvent yields 0.22 g (89%) of the compound of formula (116). The aqueous phase is adjusted to pH=1-2 with 2N hydrochloric acid and extracted with ethyl acetate. After removal of the solvent, 0.41 g (76%) of the desired compound of formula (117) is obtained in the form of a white solid.

### EXAMPLE 6

a) Hydrogenation

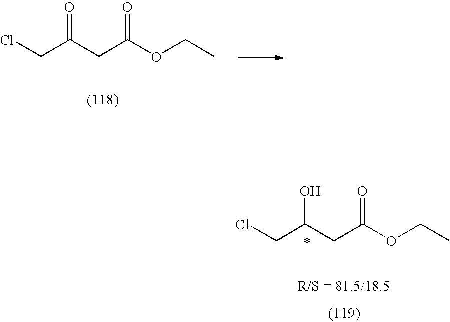

The hydrogenation is carried out analogously to the instructions in Example 1a).

b) Enzymatic Separation

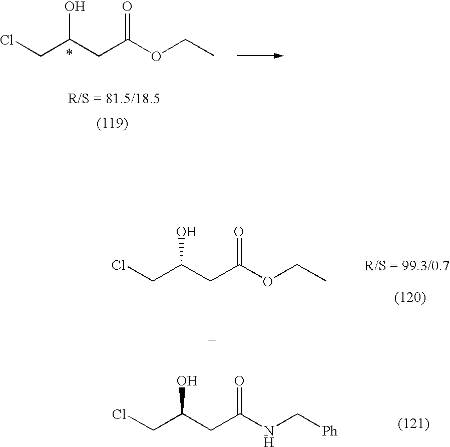

4.0 g of the compound of formula (119) obtainable as indicated in a) are dissolved in 32 ml of dioxane together with 0.55 g of benzylamine. 1.44 g of Novozym 435 (NOVO) are added thereto and the mixture is shaken vigorously at room temperature. After 1.25 hours, the protein is filtered off and the filtrate is stirred with Dowex (acid). After filtration and removal of the solvent, the product is obtained from the resulting residue by distillation. 2.95 g (91%) of the desired compound of formula (120) are obtained. The immobilised enzyme can be re-used repeatedly without loss of activity.

¹H-NMR(CDCl₀₋₃) of the compound of formula (120): 4.25 (m, 1H), 4.17 (q, J=7.33 Hz, 2H), 3.59 (m, 1H), 2.62 (m, 1H), 1.2 (t, J=7.33 Hz, 3H).

By means of HPLC (Chiracel OD), a ratio of R/S=99.3/0.7 is determined for the compound of formula (120).

Retention time R isomer: 10.32 min;

Retention time S isomer: 7.32 min.

### EXAMPLE 7

a) Hydrogenation

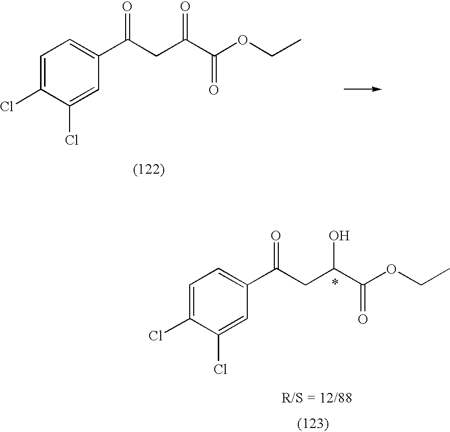

The hydrogenation is carried out analogously to the instructions in Example 1a).

b) Acetylation

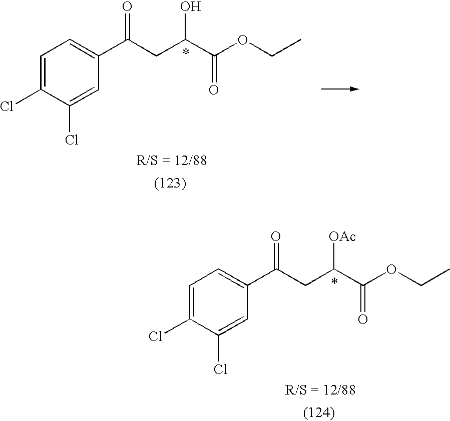

The acetylation is carried out analogously to the instructions in Example 2b).

The acetylated enantiomers are analysed on Chiracel AD. The acetate is obtained quantitatively.

Retention time R isomer: 17.47 min;

Retention time S isomer: 18.96 min.

¹H-NMR(CDCl₃) of the compound of formula (124): 8.06 (d, J=2.05 Hz, 1H), 7.81 (dd, J=2.35 Hz and 8.50 Hz; 1H), 7.60 (d, J=8.50 Hz; 1H), 5.69 (dd, J=4.10 Hz and 2.64 Hz; 1H), 4.27 (q, J=7.33 Hz; 2H), 3.56 (dd, J=4.10 und 17.59 Hz; 1H), 3.50 (dd, J=2.64 Hz and 17.59 Hz; 1H), 2.14 (s, 3H), 1.32 (t, J=7.33 Hz, 3H).

¹³C-NMR(CDCl₃) of the compound of formula (124): 14.3; 20.8; 39.9; 62.2; 68.0; 127.4; 130.4; 131.2; 133.8; 136.0; 138.5; 169.7; 170.1; 193.1.

c) Enzymatic Separation

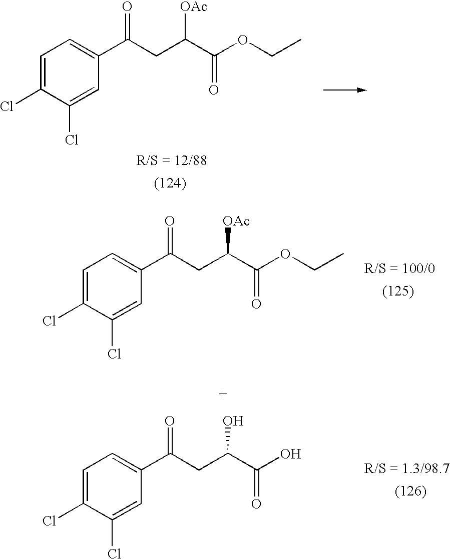

1.0 g of the compound of formula (124) obtainable according to Steps a) and b) is suspended in 40 ml of a mixture of hexane/toluene (ratio by volume 4/1) together with 20 ml of 1M phosphate buffer (pH=7). 190 mg of lipase PS (AMANO) are added thereto and the mixture is shaken at room temperature. After about 24 hours, the protein is filtered off over Celite. Dilution is effected with diethyl ether or ethyl acetate and the aqueous phase is separated off from the organic phase. The organic phase is then washed with saturated sodium chloride solution. Concentration of the organic phase by evaporation yields 0.13 g (100%) of the compound of formula (126). The aqueous phase is adjusted to pH=1-2 with 2N hydrochloric acid and extracted with ethyl acetate. After removal of the solvent, 0.59 g (79%) of the desired compound of formula (126) is obtained in the form of a white solid.

According to HPLC data, the re-isolated compound of formula (125) consists of 100% R isomer.

The R/S ratio of the enantiomers of the compound of formula (126) is determined by HPLC after esterification of a sample with ethanol (R/S=1.3/98.7).

NMR data of the compound of formula (126):

¹H-NMR(CDCl₃): 7.99 (d, J=2.05 Hz, 1H), 7.74 (dd, J=2.35 Hz and 8.50 Hz; 1H), 7.52 (d, J=8.50 Hz; 1H), 4.64 (m, 1H), 4.24 (q, J=7.33 Hz; 2H), 3.43 (dd, J=4.10 and 17.59 Hz; 1H), 3.38 (dd, J=2.64 Hz and 17.59 Hz; 1H), 1.26 (t, J=7.33 Hz, 3H).

¹³C-NMR(CDCl₃): 14.3; 42.4; 62.2; 67.2; 127.4; 130.4; 131.0; 133.6; 136.3; 138.4; 173.8; 195.4.

What is claimed is:

1. A process for the preparation of a compound of formula 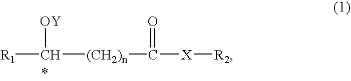
wherein
R₁ is unsubstituted or substituted C₁-C₈alkyl or a radical of formula —COOR₃, wherein R₃ is hydrogen or unsubstituted or substituted C₁-C₈alkyl,
R₂ is hydrogen or unsubstituted or substituted C₁-C₈alkyl,
X is the radical —O— or —NH—,
Y is hydrogen or an acyl or silyl radical,
n is the number 0, 1 or 2, and
the chiral carbon atom denoted by the symbol * in the compound of formula (1) is predominantly in pure form in either the R or S configuration,
in which process a compound of formula 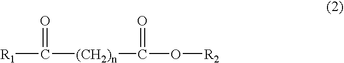
is converted by enantioselective hydrogenation and, where appropriate, introduction of the radical Y to form an enantiomeric mixture, enriched with one of the enantiomers (R or S configuration), of the compound of formula 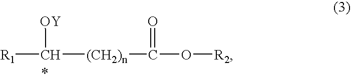
and the enantiomeric mixture is separated by enzymatic stereoselective hydrolysis, alcoholysis, aminolysis or ammonolysis;
and in the case of the preparation of compounds of formula (1) wherein X is the radical —NH—, the resolution is effected by enzymatic stereoselective aminolysis or ammonolysis in the presence of a compound of formula NH₂—R₂′, wherein R₂′ is as defined above for R₂.

2. A process according to claim 1, in which,
R₁ is C₁-C₈alkyl that is unsubstituted or substituted by halogen or by phenyl or benzoyl that are unsubstituted or further substituted by C₁-C₄alkyl, C₁-C₄alkoxy, C₁-C₄alkylamino,
C₁-C₄alkanoyl, amino, nitro or by halogen; or
R₁ is a radical of formula —COOR₃ and
R₃ is hydrogen or unsubstituted or phenyl-substituted C₁-C₈alkyl, the phenyl radical being unsubstituted or further substituted by C₁-C₄alkyl, C₁-C₄alkoxy, C₁-C₄alkylamino, C₁-C₄-alkanoyl, amino, nitro or by halogen.

3. A process according to claim 1 or claim 2, in which
R₁ is C₁-C₄alkyl that is unsubstituted or substituted by halogen or by phenyl or benzoyl that are unsubstituted or further substituted by C₁-C₄alkyl, C₁-C₄alkoxy or by halogen.

4. A process according to any one of claims 1 to 3, in which
R₂ is hydrogen or unsubstituted or phenyl-substituted C₁-C₈alkyl, the phenyl radical being unsubstituted or further substituted by C₁-C₄alkyl, C₁-C₄alkoxy, C₁-C₄alkylamino, C₁-C₄-alkanoyl, amino, nitro or by halogen.

5. A process according to any one of claims 1 to 4, in which
R₂ is C₁-C₄alkyl or benzyl.

6. A process according to any one of claims 1 to 5, in which
X is the radical —O—.

7. A process according to any one of claims 1 to 6, in which
Y is a radical of the formula —C(O)—R₄ or —Si(R₅)₃, wherein R₄ and R₅ are unsubstituted or phenyl-substituted C₁-C₈alkyl.

8. A process according to any one of claims 1 to 7, in which
n is the number 0 or 1, especially the number 1.

9. A process according to any one of claims 1 to 8, in which
the enantioselective hydrogenation is carried out using platinum as catalyst in the presence of a chiral modifier.

10. A process according to any one of claims 1 to 9, in which
the enantioselective hydrogenation is carried out using platinum as catalyst in the presence of a cinchona alkaloid as chiral modifier.

11. A process according to any one of claims 1 to 10, in which
an enantiomeric mixture, enriched with one of the enantiomers, of a compound of formula (3) wherein R₂ is unsubstituted or substituted C₁-C₈alkyl is converted by enzymatic hydrolysis to form a mixture of the compounds of formulae 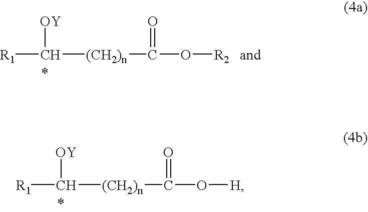
wherein
R₂ is as defined above,
R₁, Y and n are as defined in claim 1, and
one of the compounds of formulae (4a) and (4b) is in the R configuration and the other of the compounds of formulae (4a) and (4b) is in the S configuration.

12. A process according to any one of claims 1 to 10, in which
an enantiomeric mixture, enriched with one of the enantiomers, of a compound of formula (3) wherein Y is an acyl radical is converted by enzymatic hydrolysis to form a mixture of the compounds of formulae 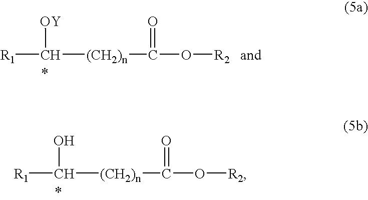
wherein
Y is as defined above and
R₁, R₂ and n are as defined in claim 1, and one of the compounds of formulae (5a) and (5b) is In the R configuration and the other of the compounds of formulae (5a) and (5b) is in the S configuration.

13. A process according to any one of claims 1 to 10, in which
an enantiomeric mixture, enriched with one of the enantiomers, of a compound of formula (3) wherein R₂ is unsubstituted or substituted C₁-C₈alkyl and Y is an acyl radical is converted by enzymatic hydrolysis to form a mixture of the compounds of formulae 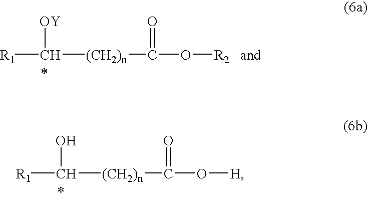
wherein
R₂ and Y are as defined above,
R₁ and n are as defined in claim 1, and
one of the compounds of formulae (6a) and (6b) is in the R configuration and the other of the compounds of formulae (6a) and (6b) is in the S configuration.

14. A process according to any one of claims 1 to 10, in which
an enantiomeric mixture, enriched with one of the enantiomers, of a compound of formula (3) is converted by enzymatic aminolysis or ammonolysis in the presence of a compound of formula NH₂—R₂′ to form a mixture of the compounds of formulae 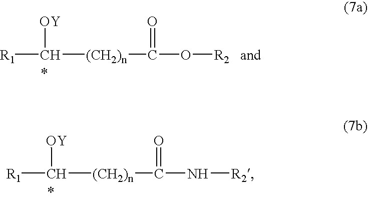
wherein
R₁, R₂, R₂′, Y and n are as defined in claim 1, and
one of the compounds of formulae (7a) and (7b) is in the R configuration and the other of the compounds of formulae (7a) and (7b) is in the S configuration.

15. A process according to any one of claims 1 to 14, in which
the enantiomeric mixture, enriched with one of the enantiomers, of the compound of formula (3) has an enantiomeric distribution of from 65/35 to 95/5, especially from 70/30 to 95/5, in favour of the R or S configuration.

16. A process according to claim 15, in which
the enantiomeric distribution is from 80/20 to 95/5 in favour of the R or S configuration.
